(19) United States
(12) Patent Application Publication
(10) Pub. No.: US 2012/0116278 A1
(43) Pub. Date: May 10, 2012

(54) Bandage Pack and Method of Packaging

(76) Inventor: Mark Louis Donald, Norfolk, VA (US)

(21) Appl. No.: 13/229,830

(22) Filed: Sep. 12, 2011

Related U.S. Application Data
(60) Provisional application No. 61/381,506, filed on Sep. 10, 2010.

(51) Int. Cl. A61F 15/00; B65B 1/04; A61F 13/02

(52) U.S. Cl. 602/42; 206/440; 53/467

(57) ABSTRACT

Articles of manufacture, methods for packaging and using bandages are described including an s-folded, or “fan folded”, bandage that is contained in a housing including an opening and a first strip of securing material proximate to the opening. The s-folded bandage may be substantially contained within the housing and may extend at least partially though the opening. The bandage may include a second strip of securing material that is configured to secure with the first strip of securing material. The second strip of securing material may include a hook material that is configured to engage with loops of the first strip of securing material and/or a bandage material of the bandage. The second strip of securing material may be folded over an end of the bandage. Aspects of the invention may provide for a low-profile bandage pack that is relatively convenient to carry and use.

### CROSS-REFERENCES TO RELATED APPLICATIONS

This application claims priority to and benefit under 35 U.S.C. §119(e) to U.S. Provisional Application No. 61/381,506, filed Sep. 10, 2010, the disclosure of which is herein expressly incorporated by reference in its entirety.

### BACKGROUND OF THE INVENTION

1. Field of the Invention

The invention relates generally to articles of manufacture including bandages, methods of packaging bandages and also finds applicability in methods of applying bandages. Aspects of the invention are particularly well suited for use in special application bandage packs, such as those with a low-profile package and those used for rapid application to a patient, such as to secure trauma dressings.

b 2. Related Art

A bandage is a piece of material used typically to support a dressing, a splint, and/or a part of a patient's body. Bandages are available in a wide range of types, that may perform different functions and be particularly suited for different applications. For example, common gauze bandages may be made of a light cotton, or other gauze material, and be used to hold a dressing in place, whereas a compression bandage may be made from combinations of materials that provide light to moderate compression when applied adhesive to the skin of, and/or wrapping around, a body part of the patient.

Different types of bandages are more or less suited to particular packaging techniques based on such factors as the material, size, and intended use of the bandage. Commonly, bandages are rolled in a circular wrap that results in a relatively high profile (i.e. the roll thickness) compared to the length of the bandage.

Due to the variety of bandages that may be needed, and the size and number of bandage packs required, medical personnel, such as combat medics, EMT's, etc., are concerned with the size as well as the ease of use of the bandages themselves. This is becoming even more true with respect to military personnel, and the like, that may be required to carry their own supply of bandages, along with an ever-increasing assortment of tactical equipment. For example, a soldier may be more likely to actually carry a bandage that is easy to include on his person and that is easy to use. Whereas, a bandage pack that is bulky and inefficient to use in situations where they are typically needed may be left behind, and therefore not be available at a critical time when it is needed for potentially life-saving treatment. This concern applies to other professions in which readily accessible bandages are a requirement, such as law enforcement, firefighters, and the like.

Bandages may also be typically sealed in a packaging, such as a shrink wrap and the like, that protect the bandage from the elements, such as water and dirt. However, such packaging may also make the bandage difficult to access in tactical environments that may include low-light to dark conditions, a user's hands and/or the bandage packing being wet, and a user having otherwise limited use of their hands, such as debilitating hand or arm injuries, and/or having to apply a dressing and bandage to one's own arm. Such packaging, and the related roll configurations, can also present problems in controlling the bandage during application as the packaging is typically discarded upon opening, leaving the user with nothing to control the bandage other than physically gripping the bandage itself. Often times, because a user's hands are engaged with applying the bandage, the unused portion of the bandage roll may be dropped and allowed to unravel.

In light of the above, there are still problems and disadvantages associated with the known methods of bandage packaging, and the related methods of bandage application, particularly in tactical environments, including bulkiness, readiness and ease of use, durability and the like.

### BRIEF SUMMARY OF THE INVENTION

The invention provides articles of manufacture, methods for packaging and using bandages including an s-folded, or “fan folded”, bandage that is contained in a housing including an opening and a first strip of securing material proximate to the opening. The s-folded bandage may be substantially contained within the housing and may extend at least partially though the opening. In embodiments, the bandage may include a second strip of securing material that is configured to secure with the first strip of securing material.

In embodiments, the second strip of securing material may include a hook material that is configured to engage with loops of the first strip of securing material and/or a bandage material of the bandage. The second strip of securing material may be folded over an end of the bandage. In other embodiments, the first strip of securing material on the housing may be configured to engage with and secure a bandage material of the bandage.

In embodiments, the bandage pack may include means for distributing a pressure applied to a top of the housing across an area of the bandage that is larger than an area of the applied pressure. The housing comprises a wall material with a stiffness similar to impregnated cardboard, nylons and/or plastic sheets of approximately 0.5 mm-2 mm. According to embodiments, the housing may be manufactured from a material having a desired stiffness, such as, for example, a nylon or plastic material. The housing may be at least partly assembled around the bandage, such as by folding walls, and/or a top surface, of the housing around the bandage. The housing may be fixed around the bandage, such as by cementing or heat-treating the material of the housing to itself, and/or providing a wrapping material around the housing, etc.

In embodiments, the bandage may include an elastic wrap, and/or may be made of woven fabrics containing elastomeric filaments interspersed among non-elastomeric filler yarns, elastic non-woven fabrics of synthetic fibers, and other materials and combinations of materials known in the art. In other embodiments, the bandage may be a substantially inelastic material, such as nylon and/or spun fiber yarns such as cotton, viscose, polyester, cotton/viscose or cotton/polyester mixtures and other materials and combinations of materials known in the art.

In embodiments, the bandage may be about 12 feet long and/or about 4 inches wide.

According to other aspects of the invention, embodiments may include a method of packaging a bandage including providing a housing including an opening and a first strip of securing material proximate to the opening. An s-folded bandage may be placed substantially within the housing with an end of the bandage extending at least partially though the opening. In embodiments, the housing may be fixed around the bandage, such as by cementing or heat-treating the material of the housing to itself, and/or providing a wrapping material around the housing, etc.

Embodiments may also include removably securing an end of the bandage to the first strip of securing material via a second strip of securing material attached to the end of the bandage. Embodiments may include attaching the second strip of securing material to the end of the bandage such as, for example, by folding the second strip of securing material over the end of the bandage.

According to other aspects of the invention, embodiments may include a method of applying a bandage, such as those bandages and bandage packs described herein, including releasing an end of a bandage from engagement with a first strip of securing material proximate to an opening in a housing containing the bandage. Methods may include applying the bandage to a patient, including wrapping the bandage around a part of the patient's body and securing the end of the bandage to another portion of the bandage via a second strip of securing material attached to the end of the bandage that was used to engage with the first strip of securing material.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention claimed. The detailed description and the specific examples, however, indicate only preferred embodiments of the invention. Various changes and modifications within the spirit and scope of the invention will become apparent to those skilled in the art from this detailed description.

### BRIEF DESCRIPTION OF THE DRAWINGS

The accompanying drawings, which are included to provide a further understanding of the invention, are incorporated in and constitute a part of this specification, illustrate embodiments of the invention and together with the detailed description serve to explain the principles of the invention. No attempt is made to show structural details of the invention in more detail than may be necessary for a fundamental understanding of the invention and various ways in which it may be practiced. In the drawings:

FIG. 1 depicts an interior side of a partially assembled exemplary bandage housing according to the principles of the invention.

FIG. 2 depicts an exterior side of a partially assembled exemplary bandage housing according to the principles of the invention.

FIG. 3 depicts an exterior side of another partially assembled exemplary bandage housing according to the principles of the invention.

FIG. 4 depicts a side view of a an exemplary s-folded bandage according to the principles of the invention.

FIG. 5 depicts a side view of an exemplary bandage pack according to the principles of the invention.

FIG. 6 depicts an isometric view of an exemplary bandage pack and partially extended bandage according to the principles of the invention.

FIG. 7 depicts aspects of using an exemplary bandage pack and partially extended bandage according to the principles of the invention.

### DETAILED DESCRIPTION OF THE INVENTION

It is understood that the invention is not limited to the particular methodology, protocols, and reagents, etc., described herein, as these may vary as the skilled artisan will recognize. It is also to be understood that the terminology used herein is used for the purpose of describing particular embodiments only, and is not intended to limit the scope of the invention. It also is be noted that as used herein and in the appended claims, the singular forms “a,” “an,” and “the” include the plural reference unless the context clearly dictates otherwise. Thus, for example, a reference to “a lesion” is a reference to one or more lesions and equivalents thereof known to those skilled in the art.

Unless defined otherwise, all technical and scientific terms used herein have the same meanings as commonly understood by one of ordinary skill in the art to which the invention pertains. The embodiments of the invention and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the invention. The examples used herein are intended merely to facilitate an understanding of ways in which the invention may be practiced and to further enable those of skill in the art to practice the embodiments of the invention. Accordingly, the examples and embodiments herein should not be construed as limiting the scope of the invention, which is defined solely by the appended claims and applicable law. Moreover, it is noted that like reference numerals reference similar parts throughout the several views of the drawings.

Moreover, provided immediately below is a “Definition” section, where certain terms related to the invention are defined specifically. Particular methods, devices, and materials are described, although any methods and materials similar or equivalent to those described herein can be used in the practice or testing of the invention. All references referred to herein are incorporated by reference herein in their entirety.

The term “bandage” as used herein refers to pieces of material used typically to protect, immobilize, compress, or support a wound or injured body part, and/or support a dressing, a splint, or similar medical device. Bandages are distinct from, but may by packaged with or attached to, dressings, that are typically applied directly to a wound. Bandages may be elastic or inelastic.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Thus, for example, the present method of “treating” individuals afflicted with conditions requiring bandaging, as the term “treating” is used herein, encompasses treatment of conditions that suggest the applicability of bandages, with or without splints, dressings and other medical devices typically secured with bandages, to the symptomatic individual.

The term “patient” as in treatment of “a patient” refers to a mammalian individual afflicted with or prone to a condition, disease or disorder as specified herein, and includes both humans and animals.

The following preferred embodiments may be described in the context of exemplary bandage packs for ease of description and understanding. However, the invention is not limited to the specifically described bandage packs and methods, and may be adapted to various clinical applications without departing from the overall scope of the invention.

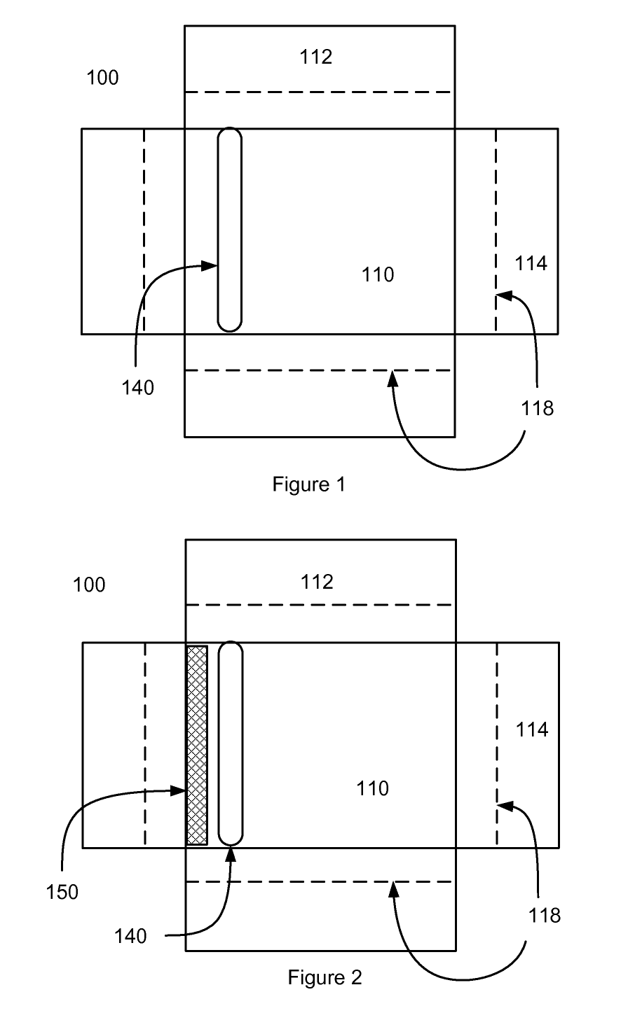

FIG. 1 depicts aspects of an interior surface of an exemplary partially constructed bandage pack. The “interior surface” may be understood as a side of the sheet that may be used to form at least a part of an inner surface of an assembled bandage pack. As shown in FIG. 1, an exemplary bandage pack housing 100 may include a substantially flat sheet 110 of housing material. As described further below, the housing material may preferably include nylon, plastics, card stock, and other materials with sufficient stiffness to provide a resistance to deformation of the assembled bandage pack. At least part of the interior surface may also have additional materials or treatments applied to it, such as, for example, providing a friction surface to further resist the bandage being pulled out of the package when an external pressure is applied to the housing. The housing material may also be advantageously composed of materials that are suited to binding to one another through cementing, heat treatment and other methods known in the art. Although shown as a sheet, the materials comprising the top, bottom and/or wall of the pack may be configured in other ways depending on the desired characteristics of the pack, such as reducing a weight and/or total materials, by leaving voids in non-essential portions of the housing, using webbing and other structural configurations, and the like.

As further shown in FIG. 1, sheet 110 may be formed with, or modified to include, sets of wall and top portions 112 and 114 that may be folded to form an enclosure for a bandage. The interior surface of wall/top portions 112 and 114, as shown in FIG. 1, may be configured with preformed fold lines and/or creases 118 to ease in an assembly process.

Sheet 110 may be formed with, or modified to include, an opening 140. Opening 140 may be dimensioned to allow a bandage to pass through the opening. In embodiments, the opening 140 may include additional material, not shown, to provide a better seal to the interior of the bandage pack and/or resist the movement of the bandage through the opening 140, such as, for example, flexible flaps, a rough material, adhesives, and the like. It should be noted that, although shown as a substantially unitary construction 100, the bandage housing may include multiple sheets of materials in different shapes and configurations without departing from the intended scope of the invention. Further aspects of the exemplary housing 100 are described with reference to FIG. 2.

As shown in FIG. 2, the sheet 110 of housing material may include additional features on an opposite exterior side, that is, a side of the sheet that may be used to form at least a part of an outer surface of an assembled bandage pack. As with the interior side, the exterior side of sheet 110 may be formed with, or modified to include, sets of wall and top portions 112 and 114, and an opening 140. Preformed fold lines 118 may be included on either or both of the interior and exterior sides. The exterior side of opening 140 may be dimensioned to allow a bandage to pass through the opening, and may include additional material, not shown, to provide a better seal to the interior of the bandage pack and/or resist the movement of the bandage through the opening 140, such as, for example, flexible flaps, a rough material, adhesives, and the like. At least part of the exterior surface may also have additional materials or treatments applied to it, such as, for example, providing a moisture resistant material or coating to improve the durability of the housing material.

The sheet 110 may further include a strip 150 of securing material, such as, for example hook, loop, adhesive and other materials that may be used to temporarily secure a part of a bandage to the housing. According to the embodiment depicted in FIG. 2, the strip 150 of securing material includes a hook material. As discussed further below, embodiments may include configuring the strip 150 of securing material to engage with a material of the bandage and/or a second strip of securing material that is attached to at least part of the bandage. In various embodiments, the second strip of securing material may be a different material from the securing material on the housing, such as using complimentary hook and loop material, or may be similar material to the securing material on the housing, such as adhesives, etc.

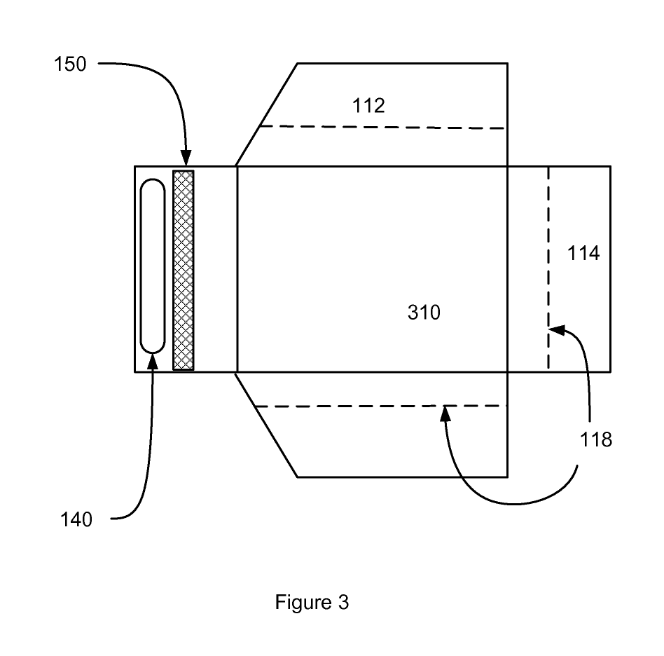

As further shown in FIG. 2, the strip 150 may be positioned proximate to the opening 140 such that at least a part of a bandage extending from the opening may be temporarily secured to the strip 150. For example, the strip 150 may be positioned at a distance from, or abutting, the opening 140. The dimensions of the strip 150 may vary depending on the type of securing material and a desired retaining force to be applied to the bandage. In embodiments, the strip 150 may be a low-profile hook material with a width of less than 1 cm, e.g. approximately 7 mm, and a length substantially corresponding to a width of the bandage contained in the housing. An alternative configuration of a housing material is shown in FIG. 3.

As shown in FIG. 3, a sheet 310 may include similar features to those described in FIGS. 1 and 2, for example, wall and top portions 112 and 114, an opening 140, preformed fold lines 118, and a strip 150 of securing material. In the embodiment shown in FIG. 3, the opening 140 and strip 150 are included on a portion of the sheet that may be folded to form an assembled housing. In embodiments, a sheet 310 may include one or more additional openings, which may be similar to opening 140, that allow, for example, the bandage to be accessed from either end of the bandage. For example, an opening (not shown) may be provided in a bottom portion of the bandage pack that allows the tail end of the bandage within the pack to extend through the pack, and/or to be accessed independently of the leading edge of the bandage.

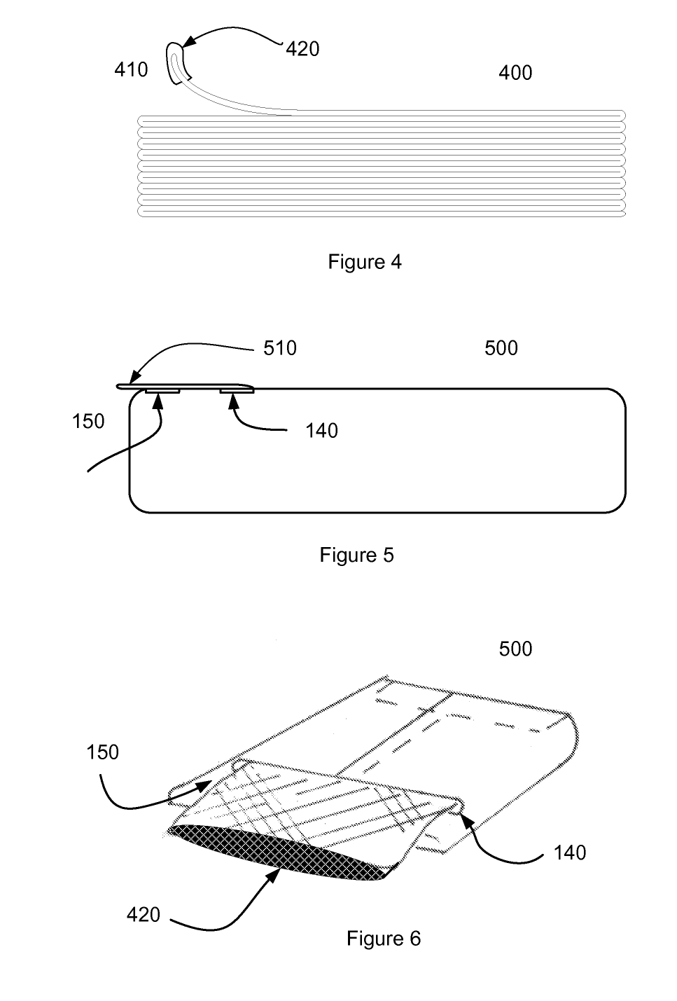

FIG. 4 depicts aspects of an exemplary bandage according to principles of the invention. As shown in FIG. 4, a bandage 400 may be s-folded, or “fan folded”, to form a stack that is relatively low-profile with respect to a length of the bandage. The bandage may be dimensioned as needed according to applications known in the art, for example, a compression bandage of about 12 feet long and about 4 inches wide may be provided. The dimensions of an s-folded 12 foot bandage may be approximately 6 inches long and ½ inches thick. The bandage may be formed from materials known in the art, such as those effective for elastic, compression and/or inelastic bandages. A leading edge 410 of the bandage 400 may include a strip 420 of securing material, such as hook, loop, and adhesive materials. In the embodiment shown in FIG. 4, the strip 420 is a loop material folded over the end 410 so that a similar loop material is presented on both sides of the bandage 400 at the end 410. As used herein, “sides” of the bandage may be understood as the top and bottom surfaces of the bandage as it would typically be applied to a patient. Other configurations are also contemplated, such as, for example, including a strip of securing material on an opposite end of the bandage, including a strip of securing material along a length of one side of the bandage, etc.

With regard to the embodiment depicted in FIG. 4, folding the strip 420 over the end 410 may provide benefits in manufacturing and use of the bandage. For example, folding the strip 420 over the end 410 of the bandage 400 may allow the strip to be attached to the bandage with one line of stitching (not shown) that may reduce manufacturing complexity, time and costs. Additionally, having the strip 420 on both sides of the bandage 400 at leading edge 410 may allow the leading edge to be easily secured to the housing, by the bottom of the bandage, and also allow the bandage to secure to itself, by the top of the bandage, when applied to the patient. As mentioned above, embodiments may include a strip of securing material included along a length of one side of the bandage. For example, a length of hook material may be included in at least a part of the length along a bottom of the bandage, which may be used to secure the bottom of the bandage to the top of the folded securing strip 420 when applied to a patient. Alternatively, at least a part of the strip 420 may be configured to engage with a material of the bandage itself. In embodiments, the strip 420 may be formed from a hook material that is configured to engage with a loop material on the housing and a material of the bandage.

FIG. 5 depicts a side view of an exemplary bandage pack 500 according to principles of the invention. The housing of the pack 500 may be at least partly assembled around a bandage, such as by folding walls, a top surface, and/or a bottom surface, of the housing around the bandage. The housing may be fixed around the bandage, such as by cementing or heat-treating the material of the housing to itself, providing a wrapping material around the housing, and/or other fixing techniques known in the art. As shown in FIG. 5, bandage pack 500 may include an s-folded bandage, such as shown in FIG. 4, that extends from an opening 140. According to embodiments, bandage pack 500 may have dimensions of approximately 5 inches wide and 7 inches long for a s-folded bandage with a width of approximately 4 inches, a total length of about 12 feet, and a folded length of about 6 inches.

The end 510 of the bandage may be secured to the housing of the bandage pack 500 by a securing strip 150. For example, the end 510 may include a loop material on at least a bottom side of the bandage that is configured to engage with a hook material of the strip 150. Other configurations are also contemplated including, for example, strip 150 being an adhesive or other securing material that is configured to engage with a material of the bandage.

FIG. 6 depicts an isometric view of exemplary bandage pack 500. As shown in FIG. 6, the end of the bandage may include a strip 420 that is released from engagement with a strip 150 by pulling the bandage. The bandage may then be used as needed by pulling additional material from the bandage pack 500. In use, the user may restrict the bandage from leaving the housing by applying pressure to the housing, such as squeezing the housing. As described herein, additional means for distributing the force applied to the housing and/or restricting the bandage from leaving the housing may be included in the housing. For example, the housing may be formed from a material of sufficient stiffness to distribute the compressive forces over a greater area than the area of applied force. This may be advantageous in allowing a controlled distribution of the bandage, compared to other, less stiff packaging materials, that may result in the bandage pinching when force is applied. The interior surface of the housing may also be coated, treated, or otherwise configured, to increase or decrease a friction applied to bandage as it moves inside the housing. For example, in the case of a relatively stiff housing, an interior surface of the housing may be configured with a relatively low coefficient of friction to provide a smooth pull of the bandage from the housing. On the contrary, a relatively high coefficient of friction may be desirable in applications where the user wants the ability to securely stop the bandage from further extension during application. Such features may provide advantages over bandage packages that do not contribute to the actual use of the bandage.

As can also be seen in FIG. 6, aspects of the invention may provide for a low-profile bandage pack that is relatively convenient to carry and use. For example, one or more relatively low-profile bandage packs, such as pack 500, can be placed in a pocket, or other containers, without the bulkiness typically found in a rolled bandage of similar length. Such configurations may be particularly desirable for EMT, military, law enforcement, and firefighting personnel, that carry significant amounts of equipment and/or need readily accessible and useable emergency medical supplies.

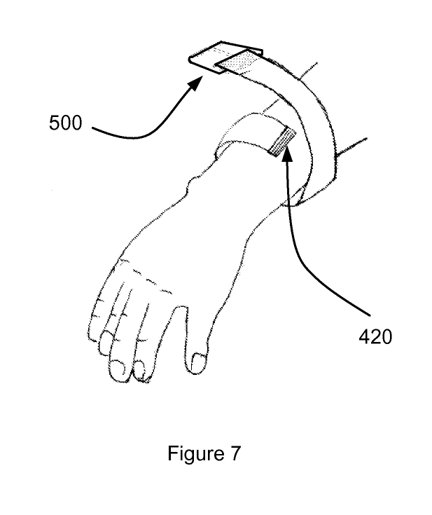

FIG. 7 depicts aspects of using an exemplary bandage pack according to principles of the invention. As shown in FIG. 7, a bandage pack 500 may be provided for use in treating a patient, such as securing a bandage to the arm of the patient. A strip 420 of securing material may be attached to the end of the bandage that may be used to secure the end of the bandage to the bandage pack 500 prior to use. When the strip 420 is detached from the pack 500, an appropriate length of the bandage may be pulled from the pack and wrapped around the arm of the patient. As the bandage is wrapped, tension may be advantageously applied, such as to provide compression for a dressing between the arm of the patient and the bandage, and a bottom side of the bandage secured to the strip 420. In embodiments, a strip of securing material (not shown) on the pack 500 may also be configured to secure the bandage from further leaving the pack.

The description given above is merely illustrative and is not meant to be an exhaustive list of all possible embodiments, applications or modifications of the invention. Thus, various modifications and variations of the described methods and systems of the invention will be apparent to those skilled in the art without departing from the scope and spirit of the invention. Although the invention has been described in connection with specific embodiments, it should be understood that the invention as claimed should not be unduly limited to such specific embodiments.

What is claimed is:

1. A bandage pack comprising:
a housing including an opening and a first strip of securing material proximate to the opening; and
an s-folded bandage substantially contained within the housing and extending at least partially though the opening, the bandage including a second strip of securing material that is configured to secure with the first strip of securing material.

2. The bandage pack of claim 1, wherein the second strip of securing material comprises a hook material that is configured to engage with loops of the first strip of securing material and a bandage material of the bandage.

3. The bandage pack of claim 1, wherein the second strip of securing material is folded over an end of the bandage.

4. The bandage pack of claim 1, further comprising means for distributing a pressure applied to a top of the housing across an area of the bandage that is larger than an area of the applied pressure.

5. The bandage pack of claim 1, wherein the housing comprises a wall material with a thickness of about 0.5 mm-2 mm.

6. The bandage pack of claim 1, wherein the bandage comprises an elastic wrap.

7. The bandage pack of claim 6, wherein the bandage wrap is about 12 feet long and about 4 inches wide.

8. A method of packaging a bandage comprising:
providing a housing including an opening and a first strip of securing material proximate to the opening;
placing an s-folded bandage substantially within the housing with an end of the bandage extending at least partially though the opening; and
removably securing the end of the bandage to the first strip of securing material via a second strip of securing material attached to the end of the bandage.

9. The method of claim 8, further comprising, prior to said placing, attaching the second strip of securing material to the end of the bandage including folding the second strip of securing material over the end of the bandage.

10. The method of claim 8, wherein the second strip of securing material comprises a hook material that is configured to engage with loops of the first strip of securing material and a bandage material of the bandage.

11. The method of claim 10, wherein the second strip of securing material is folded over an end of the bandage.

12. The method of claim 8, further comprising providing means for distributing a pressure applied to a top of the housing across an area of the bandage that is larger than an area of the applied pressure.

13. The method of claim 8, wherein the housing comprises a wall material with a thickness of about 0.5 mm-2 mm.

14. The method of claim 8, wherein the bandage comprises an elastic wrap.

15. The method of claim 14, wherein the elastic wrap is about 12 feet long and about 4 inches wide.

16. A method of applying a bandage comprising:
releasing an end of a bandage from engagement with a first strip of securing material proximate to an opening in a housing containing the bandage;
applying the bandage to a patient, including wrapping the bandage around a part of the patient's body; and
securing the end of the bandage to another portion of the bandage via a second strip of securing material attached to the end of the bandage that was used to engage with the first strip of securing material.
